(19) United States
(12) Patent Application Publication
(10) Pub. No.: US 2014/0193374 A1
(43) Pub. Date: Jul. 10, 2014

(54) METHODS AND COMPOSITIONS FOR CULTURING CELLS

(71) Applicant: ARTERIOCYTE INC., Cleveland, OH (US)

(72) Inventors: Yukang Zhao, Framingham, MA (US); Stephen Fischer, University Heights, OH (US); Anirudhasingh Sodha, Cleveland, OH (US)

(73) Assignee: ARTERIOCYTE INC., Cleveland, OH (US)

(21) Appl. No.: 14/099,769

(22) Filed: Dec. 6, 2013

Related U.S. Application Data
(60) Provisional application No. 61/734,921, filed on Dec. 7, 2012.

(51) Int. Cl. C12N 5/0789; C12N 5/078

(52) U.S. Cl. CPC C12N 5/0647; C12N 5/0641
USPC 424/93.7; 435/289.1; 435/402; 435/366; 435/377

(57) ABSTRACT

The invention provides, among other things, methods and compositions for expanding stem cells. The invention further provides methods, devices and systems for directing differentiation of expanded stem cells. The invention further provides methods, devices and systems for treating a subject with differentiated cells in a subject in need thereof.

### RELATED APPLICATIONS

This application claims the benefit of the filing date of U.S. Provisional Patent Application 61/734,921, filed Dec. 7, 2012. The entire teaching of this application is incorporated by reference herein.

### STATEMENT REGARDING FEDERALLY SPONSORED RESEARCH OR DEVELOPMENT

This invention was made with government support under 1R43HL110725-01A1 and 1R43AI092989-01 awarded by NIH. The government has certain rights in the invention.

### BACKGROUND

Hematopoietic stem cell and progenitor cells (HSPCs) have been used clinically for more than 50 years. HSPC transplants are routinely used to treat patients with cancers and other disorders of the blood and immune systems, with approximately 50,000 HSPC transplants performed annually worldwide according to the Worldwide Network for Blood and Marrow Transplantation (WBMT)¹. HSPCs are being used in an increasing number of experimental therapies as well, including therapeutic angiogenesis to treat myocardial ischemia² and gene therapy in an attempt to cure inherited blood disorders³ and increase resistance to HIV⁴. Additionally, HSPCs are being investigated as a cell source for generating terminally differentiated blood cell types including erythrocytes, natural killer cells, and platelets, after directed differentiation.

HSPCs can be obtained from bone marrow (BM), mobilized peripheral blood (MPB), and umbilical cord blood (UCB), although each source differs significantly in the number of HSPCs obtainable and their regenerative capacity^{5,6}. In the transplant setting, MPB has become a preferred source of HSPCs due to the relative ease (in most cases) of obtaining therapeutic doses of cells⁷. However, a subset of patients termed “poor mobilizers” do not respond well to plerixafor and other drugs normally used to mobilize HSPCs to peripheral blood, reducing the number of HSPCs collected and increasing the chances that an autologous HSPC transplant will fail⁸. Allogeneic HSPC transplants are a treatment option for such patients, but are considerably more complicated due to the need for identifying human leukocyte antigen (HLA)-matched grafts. UCB is an alternative source of HSPCs for allogeneic transplants that has been used in the clinic for more than twenty years⁹. UCB can be utilized in allogeneic transplants with more HLA discordance than BM and MPB¹⁰⁻¹², and UCB-derived HSPCs possess a greater proliferative capacity. Unfortunately, each unit of UCB contains a relatively low absolute number of total nucleated cells (TNCs) and HSPCs, which has limited its use primarily to pediatric transplant patients to date. Typically, the total CD34+ cell count from UCB is only about 10% of that from MPB¹³.

Regardless of the application, clinicians repeatedly see that administering larger numbers of cells increases the chances of achieving a long-term therapeutic benefit in patients versus delivering fewer cells. In the transplant setting for instance, most groups aim for an HSPC dose of at least 2.5×10⁶ CD34+ cells per kg to assure complete hematologic recoveryl^{4,15}, but several studies have shown that doses of more than 5×10⁶ CD34+ cells per kg are associated with faster engraftment and a reduced incidence of graft failure^{16,17}.

To overcome the challenges associated with insufficient HSPC doses and expand the use of HSPCs to a greater number of patients, numerous advancements are needed in the collection, processing, and delivery of HSPCs to patients. Ex vivo expansion of HSPCs is one strategy for overcoming the cell dose limitations of HSPC-based therapies that offers tremendous promise for improving clinical outcomes. The basic concept is that small numbers of HSPCs collected from patients can be expanded in culture without significant loss of HSPC phenotype and in vivo functionality. Increasing knowledge regarding the biology of HSPCs and improved methods for assaying the presence of primitive HSPCs have led to a plethora of new ex vivo expansion strategies. For instance, several groups have demonstrated improved expansion of primitive HSPCs via media addition of proteins and other molecules, including growth factor cocktails¹⁸⁻²⁰, transcriptional inhibitors²¹⁻²⁵, transcriptional activators^{26,27}, and copper chelating-compounds²⁸⁻³⁰, that modulate important HSPC signaling pathways. Other groups have demonstrated that culturing HSPCs directly on mesenchymal stem cell (MSC) feeder layers improves ex vivo expansion of primitive HSPCs³¹⁻³³, although human studies utilizing MSC feeder layers have not been convincing³⁴. Of note, researchers at the Fred Hutchinson Cancer Research Center (FHCRC) have recently developed an ex vivo expansion protocol in which UCB-derived HSPCs are cultured on a surface presenting immobilized Notch ligand Delta-1 in combination with a fibronectin fragment. The FHCRC group demonstrated that Delta-1-expanded cells, when combined with an non-manipulated UCB graft, could rapidly reconstitute myeloid cells in humans; however, the expanded cells did not achieve long-term engraftment³⁵.

One likely reason why existing strategies have fallen short in efficiently expanding primitive HSPCs is that they rely on culturing the cells directly on flat plastic surfaces (standard plastic dishes or culture bags) that are very different from the BM niche where HSPCs reside in vivo. Alternatively, electrospun nanofibers can mimic important features of the BM niche by providing a three dimensional culture surface with a very high density of surface bound functional groups that promote increased cell adhesion, among other things. Electrospun nanofibers have previously been shown to promote significant proliferation of UCB-derived HSPCs^{36,37}, but heretofore have only been available to the market in multi-well culture plates that are more suitable for research settings. The technology disclosed herein incorporates electrospun nanofibers into a closed culture system that combines the HSPC expansion benefits of the nanofibers with the sterility and convenience of a closed culture system better suited for clinical use.

### BRIEF SUMMARY OF THE INVENTION

In certain aspects, the invention provides a system for expansion, differentiation, and/or maintenance of functional cells comprising a core of one or more electrospun polymers enclosed within a closed culture device.

In certain aspects, the invention provides improved compositions and methods for the expansion and differentiation of HSPCs within a closed system. In one aspect, the inventors have found a way to mimic the natural environment where HSPCs exist within a closed system. For example, the inventors have found a way to mimic the natural bone marrow environment wherein HSPC exist naturally.

Accordingly, in one aspect, the invention provides nanofiber compositions for the expansion or differentiation of HSPCs comprising one or more electrospun polymers. In a related embodiment, an additional polymer is grafted onto the one or more electrospun polymers. In a related embodiment, the grafted polymer is derivatized. In one embodiment the polymer is derivatized with carboxylic, hydroxyl or amino groups. In another embodiment, the polymer is derivatized with a positively charged moiety. In another embodiment, the polymer is derivatized with a protein, polypeptide, peptide, or glycosaminoglycan e.g., a cell adhesion peptide or polypeptide or heparin.

In another embodiment, the polymer or polymers are selected from the group consisting synthetic polymers, natural polymers, protein engineered biopolymers or combinations thereof.

In a particular embodiment, the grafted polymer is poly(acrylic acid) (PAAc). In another particular embodiment, the electrospun polymers comprises polyethersulfone (PES).

In another embodiment, the composition of the invention has a spacer between the grafted nanofiber and the derivatized moiety. Exemplary spacers are ethylene, butylenes or hexylene moieties.

In particular embodiments of the invention, the compositions of the invention are useful for expanding or differentiating neural stem cells and embryonic stem cells in addition to HSPCs.

In other embodiments, the grafted electrospun nanofiber compositions of the invention have a diameter between 10 nm to 10 μm, preferably between 100-700 nm. In a specific embodiment, the grafted electrospun nanofiber composition of the invention comprises poly(acrylic acid) grafted on to a polyethersulfone core. In a further embodiment, this composition is derivatized. In particular embodiments the composition is derivatized by amination, with peptides or polypeptides, e.g., laminin, heparin, or cell adhesion peptides or polypeptides.

In a further embodiment, the electrospun fibers are made from a synthetic polymer, natural polymers, and random fibers and aligned fibers. In a further embodiment, the fibers can have a range of diameters, including from 10 nanometers to 10 micrometers.

In another embodiment, the electrospun composition comprises spacers between the electrospun fiber and the derivatized moiety. In exemplary embodiments, the spacers comprise ethylene, butylene or hexylene moieties.

In one particular embodiment, the grafted electrospun nanofiber composition comprises poly(acrylic acid) grafted on to a polyethersulfone core, wherein the poly (acrylic acid) is aminated.

In particular embodiments the electrospun fiber compositions are useful for the expansion or differentiation of stem cells, e.g., hematopoietic stem/progenitor cells, neural stem cells, or embryonic stem cells. In certain embodiments of the invention, the electrospun nanofiber composition of the invention has randomly oriented fibers. In alternate embodiments, the electrospun nanofiber composition of the invention has aligned fibers. In further embodiments, the grafted electrospun is produced uniaxial electrospinning, coaxial, or multi axial electrospinning.

In another embodiment, the electrospun nanofiber composition is attached directly to a culture container. In further embodiments, the nanofiber composition is attached to a flexible substrate (la poly-acrylate like PMMA, a poly-acetate like EVA, a polyester like PET, or a multi layer combination of the same) prior to incorporation within the culture container. In alternate embodiments, the nanofiber composition with substrate is attached to a rigid culture container prior to incorporation within the culture container. In these embodiments, the nanofiber composition is attached to the culture container or a substrate through chemical (e.g., adhesive) or non-chemical (e.g., thermal bonding) means.

In another embodiment of the invention, the culture container is composed of gas permeable materials. In an alternate embodiment, the culture container is composed of non-permeable materials.

In a further embodiment of the invention, the culture container has one inlet/outlet valve (in the form of tubing, spike connector, luer fittings, etc.) for culture medium and reagents injection, as well as cell harvesting. In alternate embodiments, the culture container has two, three or four inlet/outlet valves (in the form of tubing, spike connector, luer fittings, etc.) for culture medium and reagents injection, as well as cell harvesting.

In another embodiment, the nanofiber surface can be further modified through chemical or biological means to coat the surface with surface groups and/or proteins. In further embodiments, the nanofibers can be conjugated with functional molecules including amine groups, carboxyl groups, hydroxyl groups, peptides, proteins, glycosaminoglycans and carbohydrates.

In a further embodiment of the invention, the nanofiber composition is bonded to a substrate prior to attachment to the culture container. In this embodiment, the substrate can come from a number of substrates including synthetic polymers such as polystyrene. In this embodiment, the substrate can be bonded through chemical or non-chemical means.

In another embodiment of the invention, the material for cell culture container can come from classes of materials that allow for high rate of gas exchange (e.g., EVA, EVO) or minimal gas exchange (e.g., polystyrene, FEP).

In further embodiments of the invention, expanded cells can be used either directly or after further processing for research and therapeutic purposes in a variety of disease conditions where use of the expanded cells are useful, including but not limited to, bone marrow transplantation for patients with disease conditions like leukemia, anemia, and bone marrow failure. In an alternate embodiment, the expanded cells can be used directly, or further processed by cell selection, differentiation, and gene modification for research and therapeutic applications.

In another embodiment of the invention, the expanded cells can go through further differentiation processes in the system or in another system. One example of this is for the cells to differentiate into reticulocytes, which may be used for research or as an alternate for blood transfusion.

In alternate embodiments, a number of cells types can be used such as embryonic stem cells, fat cells, induced pluripotent stem cells, neural stem cells, liver primary cells, hematopoietic stem cells, progenitor cells, and mesenchymal stem cells.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A depicts adhesion of UCB-derived CD34+ cells to NANEX™ scaffold. FIG. 1B depicts TNC and CD34+ cell expansion after 10-day serum-free culture on NANEX™ and control substrates. This figure is adapted from Reference 12.

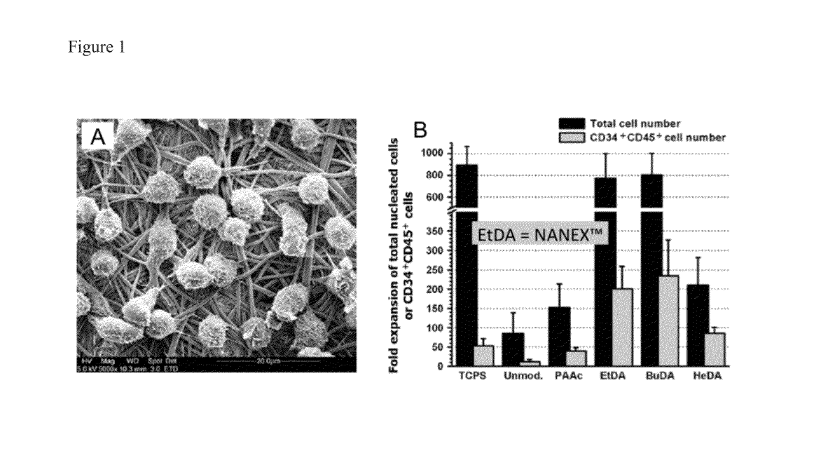

FIG. 2 depicts engraftment efficiency in bone marrow of NOD-SCID mice of unexpanded UCB-derived CD34+ cells and progeny from 600 cells expanded on NANEX™ and control substrates. This figure is adapted from Reference 12.

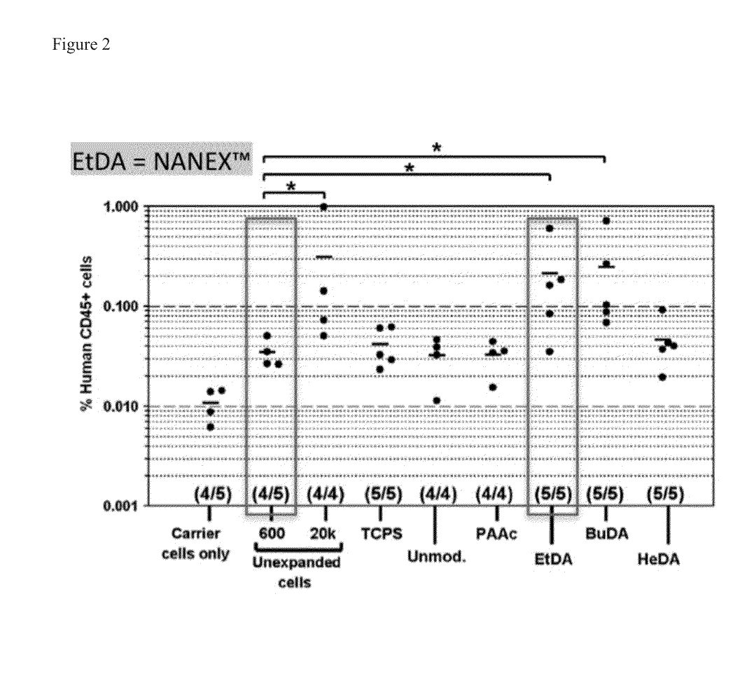

FIG. 3 depicts a culture bag embodiment of the invention.

FIG. 4 depicts the inside of a culture bag embodiment of the invention.

FIG. 5 depicts expansion of CD34+ cells in culture bag embodiment of the invention compared to traditional culture flask (TCPS) and a commercially available culture bag (VueLife® AC, American Fluoroseal Corp).

FIG. 6 depicts expansion of colony forming unit (CFU) cells in culture bag embodiment of the invention compared to traditional culture flask (TCPS).

FIG. 7 depicts a schematic of how nanofiber expansion of HSPCs is incorporated into a step-wise process for generating large numbers of differentiated erythrocytes.

FIG. 8 shows expansion results and flow cytometry analysis of cells at various time points during the erythroid differentiation stage (post-expansion in nanofiber-coated bag).

FIG. 9 shows enucleation of erythrocytes at various time points during the erythroid differentiation stage (post-expansion in nanofiber-coated bag).

### DETAILED DESCRIPTION OF THE INVENTION

### Overview

Stem cells have the potential to cure numerous disease and disorders. However, the sources of stem cells are limited. A representative example of the problem with obtaining stem cells is illustrated by human umbilical cord blood (UCB) hematopoietic stem/progenitor cells (HSPCs). HSPCs are multipotent cells that have the capacity to self-renew and differentiate into all mature blood cell types. However, a low number of HSPCs is available from sources like umbilical cord blood which limits the use of these cells to pediatric populations. Therefore, several approaches have been explored to expand HSPCs in ex vivo expansion systems, so that UCB could serve as a readily viable source of transplantable HSPCs for adult patients for the treatment of various disorders. In conventional ex vivo expansion culture, HSPCs are generally regarded as suspension cells and numerous protocols implement HSPC suspension cultures in flasks or bags in the presence of various combinations of early acting cytokines. These protocols do not produce enough HSPCs to be of clinical significance. Similar problems exist with the expansion of other types of stem cells. Accordingly, the need exists for improved methods and compositions for the expansion and differentiation of stem cells, particularly within closed culture devices that enable sterile processing of the cells for use in clinical applications.

In certain aspects, the disclosure provides a nanofiber surface within a closed culture device to mimic the human stem cell niche and promote improved expansion of functional HSPCs. By greatly increasing the number of transplantable cells, the system can overcome the limitation of the low cell numbers, and improve the clinical outcomes.

In certain aspects, the disclosure provides a fully closed system with its inner surfaces coated with one or more electrospun polymer-based scaffold. It is designed for expansion of a variety of cell lines including but not limited to, HSPCs which can be injected into patients with various disease conditions (e.g. leukemia, bone marrow failure). The system may be used for large-scale culture and expansion of other cell types for clinical or research purposes.

### Definitions

For convenience, certain terms employed in the specification, examples, and appended claims, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

### Closed Culture Device

The invention pertains to the development of a novel closed culture device for sterile expansion and differentiation of stem cells as well as methods and compositions for the expansion and differentiation of stem cells using nanofiber scaffolds incorporated within the closed culture device. Existing closed culture devices largely consist of flat plastic culture surfaces (e.g., tissue culture treated polystyrene) that do not adequately mimic the native environments where stem cells reside in vivo. The current invention represents a significant advancement in the field by providing a closed culture device that incorporates a culture substrate capable of better mimicking the native stem cell niche.

In one embodiment, an electrospun nanofiber composition is attached directly to a culture container. In further embodiments, the nanofiber composition is attached to a flexible substrate (a poly-acrylate like PMMA, a poly-acetate like EVA, a polyester like PET, or a multi layer combination of the same) prior to incorporation within the culture container. In alternate embodiments, the nanofiber composition with substrate is attached to a rigid culture container prior to incorporation within the culture container. In these embodiments, the nanofiber composition is attached to the culture container or a substrate through chemical (e.g., adhesive) or non-chemical (e.g., thermal bonding) means.

In one embodiment of the invention, the culture container is composed of gas permeable materials. In an alternate embodiment, the culture container is composed of non-permeable materials.

In a further embodiment of the invention, the culture container has one inlet/outlet valve (in the form of tubing, spike connector, luer fittings, etc.) for culture medium and reagents injection, as well as cell harvesting. In alternate embodiments, the culture container has two, three or four inlet/outlet valves (in the form of tubing, spike connector, luer fittings, etc.) for culture medium and reagents injection, as well as cell harvesting.

In another embodiment of the invention, the material for cell culture container can come from classes of materials that allow for high rate of gas exchange (e.g., EVA, EVO) or minimal gas exchange (e.g., polystyrene, FEP).

In certain embodiments, said device can allow for automated control of oxygen levels, carbon dioxide levels, temperature, pH levels, and the level of cell waste including ammonia or other ammonia related waste products referred to as ammoniac. In this embodiment, said device allows probes to be inserted within the device for continuous monitoring of oxygen, carbon dioxide, temperature, pH and accumulation of culture waste. Such probes are additionally connected to a computer terminal that analyzes the incoming data and outputs signals that control operation of pumps and valves to inject appropriate reagents into the device. Reservoirs of culture media, gas, and pH control reagents are connected via closed loops to the culture device to maintain sterile conditions during injection.

In certain embodiments of the invention, HSPCs are expanded within said device. In other embodiments, HSPCs are expanded and terminally differentiated within said device. In still other embodiments, HSPCs are expanded within said device and terminally differentiated in a separate device (e.g., standard culture bag) connected to the said device via a closed loop.

### Nanofiber Compositions

The present invention incorporates an electrospun nanofiber mesh within a novel closed culture system. In certain embodiments, the nanofiber mesh is that described in US Patent Application Publication No. 20080153163, herein incorporated by reference in its entirety.

In other embodiments, the electrospun nanofibers used in the methods and compositions of the invention can be natural or synthetic. In one embodiment, the electrospun nanofibers are comprised of natural polymers. Exemplary natural polymers include cellulose acetate (CA), chitin, chitosan, collagen, cotton, dextran, elastin, fibrinogen, gelatin, heparin, hyaluronic acid (HA), poly 3-hydroxybutyrate-co-3-hydroxyvalerate (PHBV), regenerated cellulose (RC), silk, and zein.

In one embodiment, the electrospun nanofibers are made of degradable or non-degradable synthetic polymer material. Exemplary degradable polymers include poly(c-caprolactone) (PCL), poly(ε-caprolactone-co-ethyl ethylene phosphate) (PCLEEP), poly(lactic acid) (PLA), poly(lactic-co-glycolic acid) (PLGA), poly(lactic acid-co-ε-caprolactone) (PLACL), and polydioxanone (PDO). Exemplary non-degradable polymers include poly acrylamide (PAAm), poly acrylic acid (PAA), poly acrylonitrile (PAN), poly amide (Nylon) (PA, PA-4,6, PA-6,6), poly aniline (PANI), poly benzimidazole (PBI), poly bis(2,2,2-trifluoroethoxy) phosphazene, poly butadiene (PB), poly carbonate (PC), poly ether amide (PEA), poly ether imide (PEI), poly ether sulfone (PES), poly ethylene (PE), poly ethylene-co-vinyl acetate (PEVA), poly ethylene glycol (PEG), poly ethylene oxide (PEO), poly ethylene terephthalate (PET), poly ferrocenyldimethylsilane (PFDMS), poly 2-hydroxyethyl methacrylate (HEMA), poly 4-methyl-1-pentene (TpX), poly methyl methacrylate (pMMA), poly p-phenylene terephthalamide (PPTA), poly propylene (PP), poly pyrrole (PPY), poly styrene (PS), polybisphenol-A sulfone (PSF), poly sulfonated styrene (PSS), Styrene-butadiene-styrene triblock copolymer (SBS), poly urethane (PU), poly tetrafluoro ethylene (PTFE), poly vinyl alcohol (PVA), poly vinyl carbazole, poly vinyl chloride (PVC), poly vinyl phenol (PVP), poly vinyl pyrrolidone (PVP), and poly vinylidene difluoride (PVDF). A preferred synthetic polymer is polyethersulfone (PES).

The electrospun nanofiber compositions of the invention can be made of any one of polymers identified herein. The electrospun nanofiber compositions of the invention can also be made of any combination of the polymers identified herein.

Electrospun matrices can be formed of electrospun fibers of synthetic polymers that are biologically compatible. The term “biologically compatible” includes copolymers and blends, and any other combinations of the forgoing either together or with other polymers. The use of these polymers will depend on given applications and specifications required. A more detailed discussion of these polymers and types of polymers is set forth in Brannon-Peppas, Lisa, “Polymers in Controlled Drug Delivery,” Medical Plastics and Biomaterials, November 1997, which is incorporated herein by reference.

The compounds to be electrospun can be present in the solution at any concentration that will allow electrospinning. In one embodiment, the compounds may be electrospun are present in the solution at concentrations between 0 and about 1.000 g/ml. In another embodiment, the compounds to be electrospun are present in the solution at total solution concentrations between 10-30 w/v % (100-300 mg/ml).

The compounds can be dissolved in any solvent that allows delivery of the compound to the orifice, tip of a syringe, under conditions that the compound is electrospun. Solvents useful for dissolving or suspending a material or a substance will depend on the compound.

By varying the composition of the fibers being electrospun, it will be appreciated that fibers having different physical or chemical properties may be obtained. This can be accomplished either by spinning a liquid containing a plurality of components, each of which may contribute a desired characteristic to the finished product, or by simultaneously spinning fibers of different compositions from multiple liquid sources, that are then simultaneously deposited to form a matrix. The resulting matrix comprises layers of intermingled fibers of different compounds. This plurality of layers of different materials can convey a desired characteristic to the resulting composite matrix with each different layer providing a different property, for example one layer may contribute to elasticity while another layer contributes to the mechanical strength of the composite matrix. These methods can be used to create tissues with multiple layers such as blood vessels.

The electrospun nanofiber has an ultrastructure with a three-dimensional network that supports cell expansion, growth, proliferation, and/or differentiation. This three dimensional network is similar to the environment where many of these HSPCs naturally occur, e.g., in bone marrow. The spatial distance between the fibers plays an important role in cells being able to obtain nutrients for growth as well as for allowing cell-cell interactions to occur. Thus, in various embodiments of the invention, the distance between the fibers may be about 50 nanometers, about 100 nanometers, about 150 nanometers, about 200 nanometers, about 250 nanometers, about 300 nanometers, about 350 nanometers, about 600 nanometers, about 750 nanometers, about 800 nanometers, about 850 nanometers, about 900 nanometers, about 950 nanometers, about 1000 nanometers (1 micron), 10 microns, 10 microns, 50 microns, about 100 microns, about 150 microns, about 200 microns, about 250 microns, about 300 microns, about 350 microns, about 400 microns, about 450 microns, or about 500 microns. In various embodiments the distance between the fibers may be less than 50 nanometers or greater than 500 microns and any length between the quoted ranges as well as integers.

Additionally, in various embodiments of the invention, the fibers can have a diameter of about 50 nanometers, about 100 nanometers, about 150 nanometers, about 200 nanometers, about 250 nanometers, about 300 nanometers, about 350 nanometers, about 600 nanometers, about 750 nanometers, about 800 nanometers, about 850 nanometers, about 900 nanometers, about 950 nanometers, about 1000 nanometers (1 micron), 50 microns, about 100 microns, about 150 microns, about 200 microns, about 250 microns, about 300 microns, about 350 microns, about 400 microns, about 450 microns, or about 500 microns, or the diameter may be less than 50 nanometers or greater than 500 microns and any diameter between the quoted ranges as well as integers. A preferred fiber diameter is between 100-700 nm.

The pore size in an electrospun matrix can also be controlled through manipulation of the composition of the material and the parameters of electrospinning. In some embodiments, the electrospun matrix has a pore size that is small enough to be impermeable to one or more types of cells. In one embodiment, the average pore diameter is about 500 nanometers or less. In another embodiment, the average pore diameter is about 1 micron or less. In another embodiment, the average pore diameter is about 2 microns or less. In another embodiment, the average pore diameter is about 5 microns or less. In another embodiment, the average pore diameter is about 8 microns or less. Some embodiments have pore sizes that do not impede cell infiltration. In another embodiment, the matrix has a pore size between about 0.1 and about 100 μm. In another embodiment, the matrix has a pore size between about 0.1 and about 50 μm. In another embodiment, the matrix has a pore size between about 1.0 μm and about 25 μm. In another embodiment, the matrix has a pore size between about 1.0 μm and about 5 μm.

The mechanical properties of the matrix or core will depend on the polymer molecular weight and polymer type/mixture. It will also depend on the orientation of the fibers (preferential orientation can be obtained by changing speed of a rotating or translating surface during the fiber collection process), fiber diameter and entanglement. The cross-linking of the polymer will also effect its mechanical strength after the fabrication process. The electrospun nanofiber core can be comprised of parallel or randomly oriented fibers.

In certain embodiments of the invention, a polymer is grafted onto the electrospun nanofiber core. Exemplary polymers that can be grafted onto the electrospun core include, but are not limited to, polymers having functional groups which can be initiated by free radicals, e.g., free radicals formed on the surface of the electrospun core. Exemplary grafted polymers include poly(acrylic acid) and derivatives and copolymers thereof, e.g., polymethacrylic acid and poly(acrylic acid-co-hydroxyethylmethacrylic acid), polyallylamine and derivatives and copolymers thereof.

In further embodiments of the invention the polymers grafted on the electro spun nanofiber core are derivatized. In general, the polymers are derivatized so that cells, e.g., HSPCs, are better able to interact with the compositions of the invention. In one embodiment, the polymers are derivatized to have a positive charge. In another embodiment, the polymers are derivatized to have a negative charge. Exemplary derivatives include carboxylic, hydroxyl and amino moieties.

In other embodiments, the polymers are derivatized with a biological agent, e.g., a nucleic acid, protein, polypeptide, peptide. In exemplary embodiments, the derivatized moiety is a cell adhesion peptide or heparin. In other exemplary embodiments, the derivatized moiety is a cytokine or growth factor. In certain embodiments, the polymer bound moiety will mimic the native presentation pattern of these molecules in the bone marrow niche during early hematopoiesis.

In certain embodiments, derivatized moieties are directly conjugated to the polymer fibers. In certain embodiments, multiple derivatized moieties are conjugated to the same set of fibers. In certain embodiments, multiple derivatized moieties are conjugated to different fibers. In certain embodiments, the fibers are arranged into defined patterns.

In yet further embodiments, the compositions of the invention comprise a spacer molecule between the electrospun nanofiber and the derivatized moiety. The spacer molecule can allow for improved functionality of the compositions of the invention. In exemplary embodiments, the spacer is an ethylene, propylene, butylene, or hexylene moiety.

### Expansion of HSPCs

In certain embodiments, the present invention discloses methods and processes to obtain large numbers of functional HSPCs through the ex vivo expansion of the cells within the disclosed device.

The instant methods rely on the isolation of stem cells from any of a number of sources and the subsequent use of the compositions and methods of the instant invention to expand these stem cells. Stem cells can be isolated from any of a number of sources using techniques known to those of skill in the art. For example, U.S. Pat. No. 5,061,620 describes a substantially homogeneous human hematopoietic stem cell composition and the manner of obtaining such composition.

In certain embodiments, there is at least a 200-fold expansion of HSPCs within the closed culture device. In certain embodiments, there is at least a 200-fold expansion of HSPCs in about a 10-day expansion within the closed culture device.

In certain embodiments, expansion occurs in less than 10 days of culture. In certain embodiments, expansion occurs after 10 days of culture. In certain embodiments, expansion occurs in about 10 days of culture. In certain embodiments, expansion occurs in about 12, 16, 18 20, 22, 24, 26 or 28 days of culture. In certain embodiments, expansion occurs between 1 to 10 days of culturing. In certain embodiments, expansion occurs between 2 to 10 days, 3 to 10 days, 4 to 10 days, 5 to 10 days, 6 to 10 days, 7 to 10 days, 8 to 10 days, or 9 to 10 of culturing. In certain embodiments, expansion occurs between 10-28 days of culturing. In certain embodiments, expansion occurs between 10-28 days, 10-12 days, 10-14 days, 10-16 days, 10-18 days, 10-20 days, 10-22 days, 10-24 days, or 10-26 days of culturing. In certain embodiments, expansion occurs on a scaffold. In certain embodiments, expansion occurs in culture on a nanofiber mesh and/or film. In certain embodiments, expansion occurs in a bioreactor.

In certain embodiments, cells expanded within the device demonstrate larger numbers of CD34+ cells in the final product than cells cultured using standard cultureware. In certain embodiments, cells expanded within the device demonstrate larger numbers of colony forming unit (CFU) cells than cells cultured using standard cultureware. In certain embodiments, expanded cells successfully reconstitute hematopoiesis at efficiency rates higher than cells cultured using standard cultureware.

### Differentiation of HSPCs

In certain embodiments, the present invention discloses methods and processes to obtain terminally differentiated blood cell types through the ex vivo expansion and differentiation of HSPCs.

One embodiment of the invention is to isolate HSPCs from a biologic source such as peripheral blood, umbilical cord blood, bone marrow, and embryonic fluid. Said HSPCs are applied to said device and are allowed to expand for a period of time. HSPCs expanded within the device are then terminally differentiated.

In certain embodiments, cultured cells according to the methods of the application have a significant differentiation commitment towards the myeloblast / monoblast lineage. In certain embodiments, cultured cells according to the methods of the application have a significant differentiation commitment towards the erythrocyte lineage. In certain embodiments, cultured cells according to the methods of the application have a significant differentiation commitment towards platelets.

In certain embodiments, 80% or more of nanofiber expanded cells are differentiated to erythrocyte phenotype in about 26 days in liquid culture. In certain embodiments, 80% or more, 90% or more, or 95% or more of nanofiber expanded cells are differentiated to erythrocyte phenotype in about 10, 12, 14, 16, 18, 20, 22, 24 or 28 days in liquid culture. In certain embodiments, HSPCs expanded within the device are differentiated using published methods such as those described in Koury et al. In vitro maturation of nascent reticulocytes to erythrocytes. Blood. 2005 Mar. 1; 105(5):2168-74. Epub 2004 Nov. 4; Fujimi et al. Ex vivo large-scale generation of human red blood cells from cord blood CD34+ cells by co-culturing with macrophages. Int J Hematol. 2008 May; 87(4):339-50; Neildez-Nguyen et al. Human erythroid cells produced ex vivo at large scale differentiate into red blood cells in vivo. Nat Biotechnol. 2002 May; 20(5):467-72; or Giarratana et al. Ex vivo generation of fully mature human red blood cells from HSPCs. Nat Biotechnol. 2005 January; 23(1):69-74. Epub 2004 Dec. 26, all of which are herein incorporated by reference in their entirety.

### Exemplification

The invention now being generally described, it will be more readily understood by reference to the following examples, which are included merely for purposes of illustration of certain aspects and embodiments of the present invention, and are not intended to limit the invention, as one skilled in the art would recognize from the teachings hereinabove and the following examples, that other stem cell sources and selection methods, other culture media and culture methods, other dosage and treatment schedules, and other animals and/or humans, all without limitation, can be employed, without departing from the scope of the invention as claimed.

### EXAMPLE 1

### Closed Cell Culture Bag Incorporating a Sheet of Electrospun Nanofibers

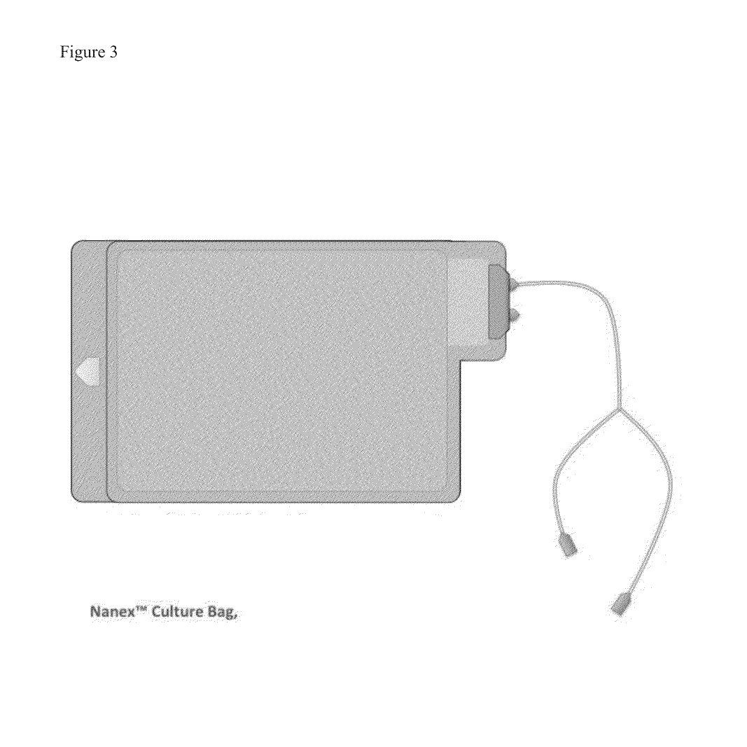

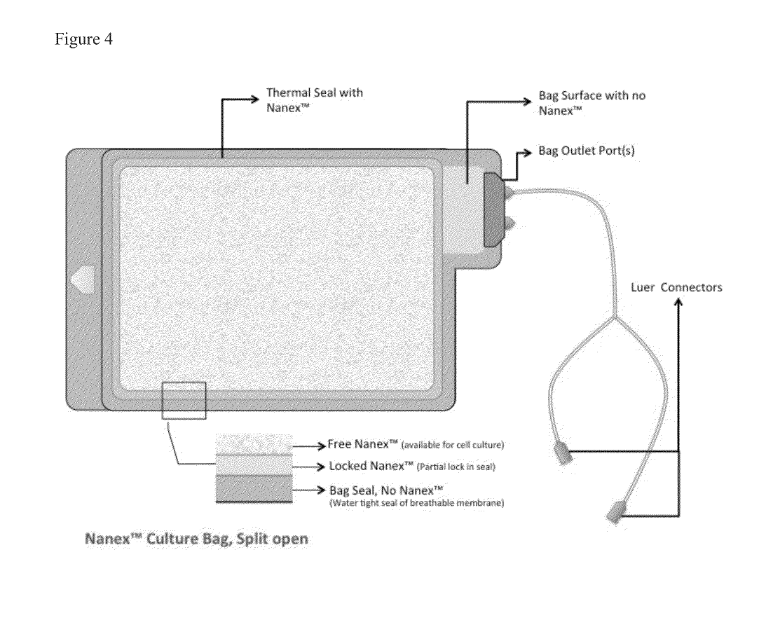

An example of this invention is to incorporate the electrospun nanofibers into a closed culture bag (see FIGS. 3 and 4). To construct such a culture bag, large sheets of electrospun polyethersulfone (PES) nanofibers were prepared and mounted onto a supportive backing material of 7.5 mil polystyrene (PS) using thermal bonding techniques. These supported PES nanofiber sheets were functionalized with a high density of amine groups. Using an adhesive, the aminated sheets were then bonded to the lower half of a sheet of gas-permeable, USP Class VI polymer in which inlet and outlet ports are embedded. The polymer sheet was then folded over and thermally sealed on three sides such that the inlet and outlet ports were located at one end of the enclosed culture bag. Transfer of media and cells into and out of the nanofiber culture bag was performed using syringes via Leur-Lok™-style connections or by sterile welding to other bags.

### EXAMPLE 2

### Expansion of Human Hematopoietic Stem and Progenitor Cells Using Electrospun Nanofiber Coated Culture Bags

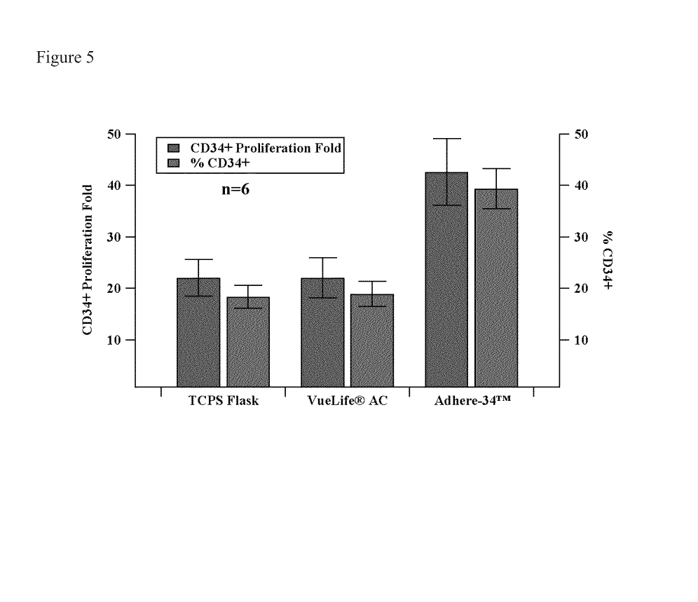

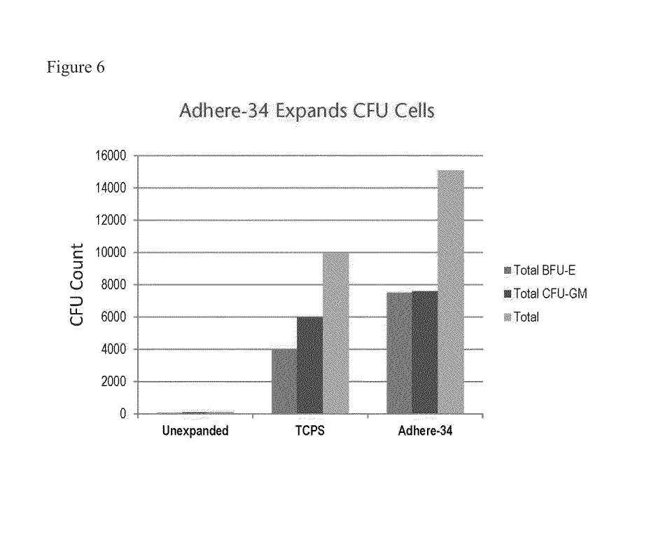

A nanofiber-coated bag of the invention can be used to expand human derived hematopoietic stem and progenitor cells (HSPCs) using various media formulations for various culture times. Human cord blood derived CD34+ cells were isolated and purified. The cells were seeded into the nanofiber cell culture bag. Cells were cultured at temperatures and atmospheric conditions that are appropriate for the type of cell being cultured. For example, CD34+ cells were cultured at a temperature of 37 degrees Celsius with an atmospheric CO₂ concentration of 5%. The cells were cultured for a number of days without medium change. In this particular example, cells were cultured for 7 days; however the culture time can be shortened or extended based on the desired ending cell population. At the end of the culture period, the culture bag was gently rocked back and forth to dislodge cells and the medium containing cells was collected. To remove remaining cells, the bags were rinsed consecutively with an appropriate rinsing agent (i.e. PBS and EDTA-based cell dissociation buffer). All the cell suspensions were combined and the cells were concentrated through centrifugation. Typically in such a culture, an expansion of at least 30-fold was achieved for CD34+ cells, with a percentage of CD34+ cells, in the final population harvested, of at least 30%. In comparison, the same CD34+ cells cultured using the same media but in a standard tissue culture polystyrene flask (TCPS Flask) or a commercially available culture bag (VueLife® AC, American Fluoroseal Corp) expand nearly 2-fold less (see FIG. 5). Additionally, total colony forming unit (CFU) cells were typically expanded about 50% more using the present invention compared to TCPS flasks (see FIG. 6).

In this particular example, human cord blood derived CD34+ cells were used. However, CD133+ cells and other hematopoietic stem and progenitor cell phenotypes can also be cultured and expanded using such nanofiber bags. In addition to human cord blood, cells derived from human bone marrow, mobilized peripheral blood, fat tissue, and fetal liver can also be cultured on such bags.

### EXAMPLE 3

### Differentiation of Nanofiber Culture Bag Expanded HSPCs Towards Erythroid Lineage.

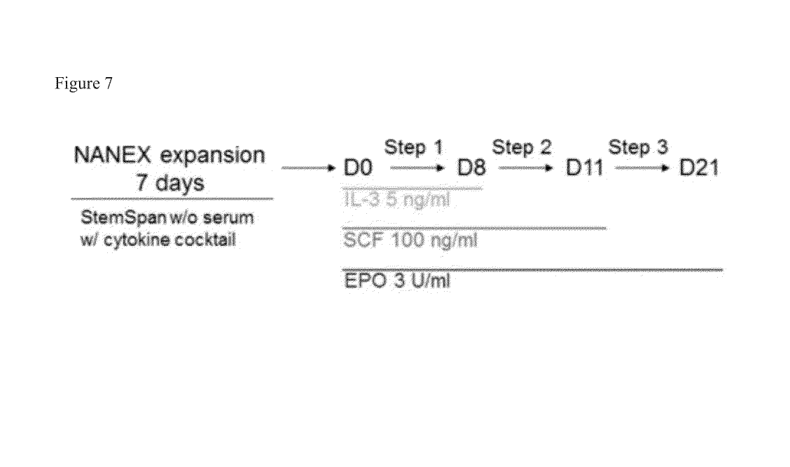

A nanofiber-coated bag of the invention can be used to expand human derived hematopoietic stem and progenitor cells (HSPCs) that can subsequently be differentiated towards various blood cell lineages. HSPCs expanded in nanofiber-coated culture bags are differentiated towards the erythroid lineage. Although several erythroid differentiation protocols are available in the literature, those protocols involving controlled feeding of particular growth factors without the need for feeder cells have been found to be particularly effective^{38,39}. A schematic of the preferred protocol is shown in FIG. 7. As shown, the first step in the process is a 7-day expansion of the HSPCs using the nanofiber-coated culture bag (referred to here as NANEX™)

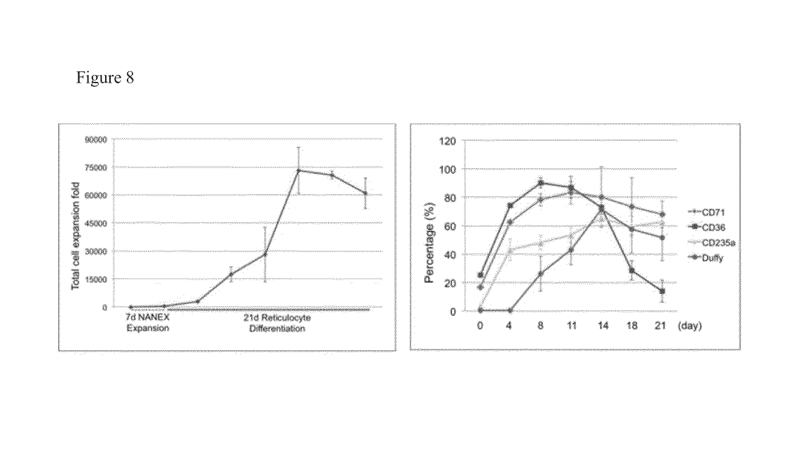

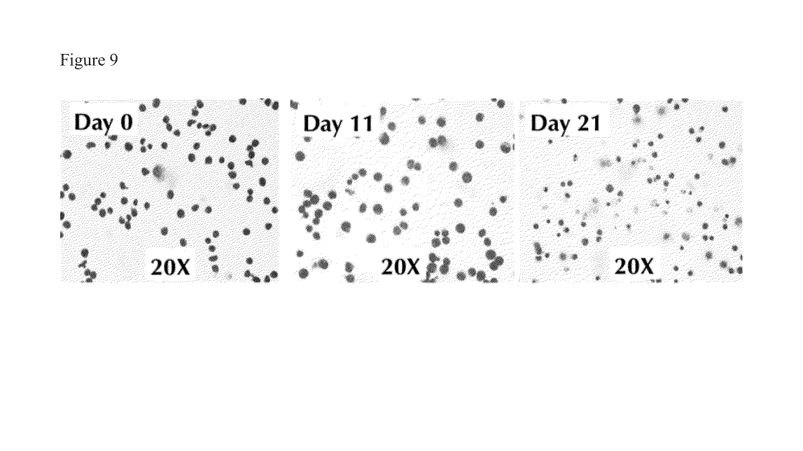

Using this approach, massive expansion of cord blood cells that appropriately expressed both early and late stage erythroid markers was achieved (FIG. 8). After 7 days of expansion using the NANEX™ system and 18-21 days of erythroid differentiation, more than 60% of cells expressed CD235a (a marker for the human erythrocyte membrane), 70% expressed CD71, and 20% expressed CD36, indicating a reticulocyte phenotype. Additionally, nearly 80% of cells were enucleated after 21 days of culture (FIG. 9).

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

### References

1 Gratwohl, A. et al. Hematopoietic stem cell transplantation: a global perspective. JAMA: the journal of the American Medical Association 303, 1617-1624, doi:10.1001/jama.2010.491 (2010).
2 Losordo, D. W. et al. Intramyocardial transplantation of autologous CD34+ stem cells for intractable angina: a phase I/IIa double-blind, randomized controlled trial. Circulation 115, 3165-3172, doi:10.1161/CIRCULATIONAHA.106.687376 (2007).
3 Gaspar, H. B. et al. Hematopoietic stem cell gene therapy for adenosine deaminase-deficient severe combined immunodeficiency leads to long-term immunological recovery and metabolic correction. Science translational medicine 3, 97ra80, doi:10.1126/scitranslmed.3002716 (2011).
4 Mitsuyasu, R. T. et al. Phase 2 gene therapy trial of an anti-HIV ribozyme in autologous CD34+ cells. Nature medicine 15, 285-292, doi:10.1038/nm.1932 (2009).
5 da Silva, C. L. et al. Differences amid bone marrow and cord blood hematopoietic stem/progenitor cell division kinetics. Journal of cellular physiology 220, 102-111, doi:10.1002/jcp.21736 (2009).
6 Wang, J. C., Doedens, M. & Dick, J. E. Primitive human hematopoietic cells are enriched in cord blood compared with adult bone marrow or mobilized peripheral blood as measured by the quantitative in vivo SCID-repopulating cell assay. Blood 89, 3919-3924 (1997).
7 Gertz, M. A. Current status of stem cell mobilization. British journal of haematology 150, 647-662, doi:10.1111/j.1365-2141.2010.08313.x (2010).
8 To, L. B., Levesque, J. P. & Herbert, K. E. How I treat patients who mobilize hematopoietic stem cells poorly. Blood 118, 4530-4540, doi:10.1182/blood-2011-06-318220 (2011).
9 Gluckman, E. et al. Hematopoietic reconstitution in a patient with Fanconi's anemia by means of umbilical-cord blood from an HLA-identical sibling. The New England journal of medicine 321, 1174-1178 (1989).
10 Liao, C. et al. Indiscernible benefit of high-resolution HLA typing in improving long-term clinical outcome of unrelated umbilical cord blood transplant. Bone marrow transplantation 40, 201-208 (2007).
11 Takahashi, S. et al. Comparative single-institute analysis of cord blood transplantation from unrelated donors with bone marrow or peripheral blood stem-cell transplants from related donors in adult patients with hematologic malignancies after myeloablative conditioning regimen. Blood 109, 1322-1330 (2007).
12 Majhail, N. S. et al. Reduced-intensity allogeneic transplant in patients older than 55 years: unrelated umbilical cord blood is safe and effective for patients without a matched related donor. Biology of blood and marrow transplantation: journal of the American Society for Blood and Marrow Transplantation 14, 282-289 (2008).
13 Soiffer, R. J. in Contemporary Hematology (ed J.E. Karp) (Humana Press, Totowa, N.J., 2008).
14 Siena, S., Schiavo, R., Pedrazzoli, P. & Carlo-Stella, C. Therapeutic relevance of CD34 cell dose in blood cell transplantation for cancer therapy. Journal of clinical oncology: official journal of the American Society of Clinical Oncology 18, 1360-1377 (2000).
15 Mavroudis, D. et al. CD34+ cell dose predicts survival, posttransplant morbidity, and rate of hematologic recovery after allogeneic marrow transplants for hematologic malignancies. Blood 88, 3223-3229 (1996).
16 Shpall, E. J., Champlin, R. & Glaspy, J. A. Effect of CD34+ peripheral blood progenitor cell dose on hematopoietic recovery. Biology of blood and marrow transplantation: journal of the American Society for Blood and Marrow Transplantation 4, 84-92 (1998).
17 Schulman, K. A., Birch, R., Zhen, B., Pania, N. & Weaver, C. H. Effect of CD34(+) cell dose on resource utilization in patients after high-dose chemotherapy with peripheral-blood stem-cell support. Journal of clinical oncology: official journal of the American Society of Clinical Oncology 17, 1227 (1999).
18 Zhang, C. C. et al. Angiopoietin-like proteins stimulate ex vivo expansion of hematopoietic stem cells. Nature medicine 12, 240-245, doi:10.1038/nm1342 (2006).
19 Tursky, M. L., Collier, F. M., Ward, A. C. & Kirkland, M. A. Systematic investigation of oxygen and growth factors in clinically valid ex vivo expansion of cord blood CD34(+) hematopoietic progenitor cells. Cytotherapy 14, 679-685, doi:10.3109/14653249.2012.666851 (2012).
20 Mohamed, A. A. et al. Ex vivo expansion of stem cells: defining optimum conditions using various cytokines. Laboratory hematology: official publication of the International Society for Laboratory Hematology 12, 86-93, doi:10.1532/LH96.05033 (2006).
21 Milhem, M. et al. Modification of hematopoietic stem cell fate by 5aza 2′deoxycytidine and trichostatin A. Blood 103, 4102-4110 (2004).
22 Araki, H. et al. Expansion of human umbilical cord blood SCID-repopulating cells using chromatin-modifying agents. Exp Hematol 34, 140-149 (2006).
23 Boitano, A. E. et al. Aryl hydrocarbon receptor antagonists promote the expansion of human hematopoietic stem cells. Science 329, 1345-1348, doi:10.1126/science.1191536 (2010).
24 Trowbridge, J. J., Xenocostas, A., Moon, R. T. & Bhatia, M. Glycogen synthase kinase-3 is an in vivo regulator of hematopoietic stem cell repopulation. Nature medicine 12, 89-98 (2006).
25 Nishino, T. et al. Ex vivo expansion of human hematopoietic stem cells by garcinol, a potent inhibitor of histone acetyltransferase. PloS one 6, e24298, doi:10.1371/journal.pone.0024298 (2011).
26 Aguila, J. R. et al. SALL4 is a robust stimulator for the expansion of hematopoietic stem cells. Blood 118, 576-585, doi:10.1182/blood-2011-01-333641 (2011).
27 Huang, C. H. et al. Purified recombinant TAT-homeobox B4 expands CD34(+) umbilical cord blood and peripheral blood progenitor cells ex vivo. Tissue engineering. Part C, Methods 16, 487-496, doi:10.1089/ten.TEC.2009.0163 (2010).
28 Peled, T. et al. Cellular copper content modulates differentiation and self-renewal in cultures of cord blood-derived CD34+ cells. Br J Haematol 116, 655-661 (2002).
29 Peled, T. et al. Pre-clinical development of cord blood-derived progenitor cell graft expanded ex vivo with cytokines and the polyamine copper chelator tetraethylenepentamine. Cytotherapy 6, 344-355 (2004).
30 de Lima, M. et al. Transplantation of ex vivo expanded cord blood cells using the copper chelator tetraethylenepentamine: a phase I/II clinical trial. Bone marrow transplantation 41, 771-778 (2008).
31 Robinson, S. N. et al. Superior ex vivo cord blood expansion following co-culture with bone marrow-derived mesenchymal stem cells. Bone marrow transplantation 37, 359-366, doi:10.1038/sj.bmt.1705258 (2006).
32 da Silva, C. L. et al. Dynamic cell-cell interactions between cord blood haematopoietic progenitors and the cellular niche are essential for the expansion of CD34+, CD34+CD38- and early lymphoid CD7+ cells. Journal of tissue engineering and regenerative medicine 4, 149-158, doi:10.1002/term.226 (2010).
33 Andrade, P. Z., dos Santos, F., Almeida-Porada, G., da Silva, C. L. & J M, S. C. Systematic delineation of optimal cytokine concentrations to expand hematopoietic stem/progenitor cells in co-culture with mesenchymal stem cells. Molecular bioSystems 6, 1207-1215, doi:10.1039/b922637k (2010).
34 De Lima, M. e. a. in Blood Vol. 116 (American Society for Hematology, Annual Meeting, Orlando, Fla., 2010).
35 Delaney, C. et al. Notch-mediated expansion of human cord blood progenitor cells capable of rapid myeloid reconstitution. Nature medicine 16, 232-236.
36 Chua, K. N. et al. Functional nanofiber scaffolds with different spacers modulate adhesion and expansion of cryopreserved umbilical cord blood hematopoietic stem/progenitor cells. Experimental hematology 35, 771-781 (2007).
37 Chua, K. N. et al. Surface-aminated electrospun nanofibers enhance adhesion and expansion of human umbilical cord blood hematopoietic stem/progenitor cells. Biomaterials 27, 6043-6051, doi:10.1016/j.biomaterials.2006.06.017 (2006).
38 Giarratana, M. C. et al. Ex vivo generation of fully mature human red blood cells from hematopoietic stem cells. Nature biotechnology 23, 69-74, doi:10.1038/nbt1047 (2005).
39 Douay, L. & Giarratana, M. C. Ex vivo generation of human red blood cells: a new advance in stem cell engineering. Methods Mol Biol 482, 127-140, doi:10.1007/978-1-59745-060-7_{—}8 (2009).

This application herein incorporates by reference the entire teachings of U.S. Patent Publication Nos. 2004/0258670, 2005/0069527 and 2009/0285892.

The practice of the present invention will employ, where appropriate and unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

1. A device for culturing cells, comprising polymeric nanofibers bonded to the interior surface of a container.

2. The device of claim 1, wherein the device has a flexible surface.

3. The device of claim 1, wherein the device has a rigid surface.

4. The device of claim 1, wherein the device has at least one inlet/outlet valve.

5. The device of claim 1, wherein the device further comprises polymeric nanofibers that are not bonded to the surface of the container.

6. The device of claim 1, wherein the device is a culture bag.

7. The device of claim 1, wherein the surface of the container comprises synthetic polymers.

8. The device of claim 7, wherein the synthetic polymers are polystyrene.

9. The device of claim 1, wherein the device is thermally sealed.

10. The device of claim 1, wherein the device is water tight.

11. The device of claim 1, wherein the device comprises a material that has a high rate of gas exchange.

12. The device of claim 1, wherein the device comprises a material that has a low rate of gas exchange.

13. The device of claim 1, wherein the device comprises at least one polymer.

14. The device of claim 13, wherein the polymer is selected from the group consisting of: polystyrene, fluorinated ethylene propylene (FEP), ethylene vinyl acetate (EVA) and EVO.

15. The device of claim 1, wherein the polymeric nanofibers are bonded directly to the interior surface of the container.

16. The device of claim 1, wherein the polymeric nanofibers are bonded to the interior surface of the container using mechanical means.

17. The device of claim 1, wherein the polymeric nanofibers are bonded to at least one substrate that is bonded to the interior surface of the container.

18. The device of claim 17, wherein the substrate is selected from the group consisting of: a poly-acrylate, a poly-acetate and a polyester.

19. The device of claim 18, wherein the substrate is selected from the group consisting of: PMMA, EVA and PET.

20. The device of claim 1, wherein the polymeric nanofibers are random nanofiber meshes or films.

21. The device of claim 1, wherein said polymeric nanofibers are aligned nanofiber meshes or films.

22. The device of claim 1, wherein said polymeric nanofibers are polyethersulfone (PES) meshes or films.

23. The device of claim 1, wherein said polymeric nanofibers are surface-conjugated with functional groups.

24. The device of claim 23, wherein said functional groups are selected from the group consisting of: hydroxyl, carboxyl, and amino groups.

25. The device of claim 1, wherein said polymeric nanofibers are surface-conjugated with fibronectin.

26. The device of claim 1, wherein the cells are stem cells.

27. The device of claim 1, wherein the cells are CD34+ cells.

28. The device of claim 1, wherein the cells are CD133+ cells.

29. A bioreactor system, comprising polymeric nanofibers bonded to the interior surface of a container and cells.

30-32. (canceled)

33. A method for expanding stem cells, comprising providing cells and culturing said cells in a culture container comprising polymeric nanofibers bonded to the interior surface.

34-64. (canceled)

65. A method of expanding stem cells, comprising:
(a) providing a population of cells including stem cells,
(b) purifying stem cells from the population,
(c) providing a bioreactor system containing polymeric nanofibers, and
(d) culturing said stem cells in said bioreactor.

66-80. (canceled)
